(19) United States
(12) Patent Application Publication
(10) Pub. No.: US 2015/0065397 A1
(43) Pub. Date: Mar. 5, 2015

(54) ASSAY ASSEMBLY

(71) Applicant: National Tsing Hua University, Hsinchu (TW)

(72) Inventors: Shih-Jie LO, Hsinchu (TW); Da-Jeng YAO, Hsinchu (TW); Kuan-Hung CHEN, Hsinchu (TW); Chao-Min CHENG, Hsinchu (TW)

(73) Assignee: National Tsing Hua University, Hsinchu (TW)

(21) Appl. No.: 14/041,275

(22) Filed: Sep. 30, 2013

(30) Foreign Application Priority Data
Sep. 3, 2013 (TW) 102131643

(51) Int. Cl. G01N 33/543

(52) U.S. Cl. CPC G01N 33/54366
USPC 506/39

(57) ABSTRACT

An assay assembly includes an assay plate. The assay plate includes at least one hydrophobic area and a plurality of hydrophilic areas defined by the hydrophobic area, wherein the hydrophilic areas are defined as a plurality of assay areas. Due to the specialized structure of the assay plate, droplets around the assay areas are drawn to the assay area by pushing force of the hydrophobic area and the pulling force of the assay area. In addition, the shaker is coupled to the assay plate and configured for shaking the assay plate thereby droplets around the assay areas are further drawn to the assay area by shaking of the shaker. The present invention may improve the error-tolerance rate of the assay plate and be used for high-throughput screening.

### BACKGROUND OF THE INVENTION

1. Field of the Invention

The present invention relates to an assay assembly, and more particularly, to an assay assembly used for high-throughput screening.

2. Description of the Prior Art

The high-throughput screening is a medical screening method going alone with combinatorial chemistry. In the end of 1990, the appearance of combinatorial chemistry has changed the method of obtaining new chemical compound. A great quantity of chemical compounds can be synthesized simultaneously in a short time with fewer steps. Under this background, the high-throughput screening technology is also developed.

The high-throughput screening technology is able to fulfill the screening of great quantity of candidate compounds in a short while. After development for one decade, it has become a mature technology applied for compound screening of the combinatorial chemistry database as well as the existing compound database.

However, the high-throughput screening technology requires measurement equipments with high precision, and this also causes the unpopularity in this technology. Hence, it is an important objective of the present invention to improve the error-tolerance rate of the assay plate and make the assay plate used for high-throughput screening.

### SUMMARY OF THE INVENTION

It is therefore an objective of the present invention to provide an assay assembly that can improve the error-tolerance rate of the assay plate and be used for high-throughput screening with the shaker and the hydrophobic and hydrophilic force.

According to one embodiment of the present invention, an assay assembly comprises an assay plate and a shaker. The assay plate is provided with at least one hydrophobic area and a plurality of hydrophilic areas defined by the hydrophobic area, wherein the hydrophilic areas are defined as a plurality of assay areas. The assay plate is coupled to the shaker. The shaker is configured for shaking the assay plate, thereby droplets around the assay areas are further drawn to the assay areas by the shaking of the shaker, the pushing force of the hydrophobic area and the pulling force of the assay areas.

Other advantages of the present invention will become apparent from the following descriptions taken in conjunction with the accompanying drawings wherein certain embodiments of the present invention are set forth by way of illustration and examples.

### BRIEF DESCRIPTION OF THE DRAWINGS

The foregoing aspects and many of the accompanying advantages of this invention will become more readily appreciated as the same becomes better understood by reference to the following detailed descriptions, when taken in conjunction with the accompanying drawings, wherein:

FIG. 1 is a schematic diagram showing the assay assembly according to one embodiment of the present invention;

FIG. 2 is a schematic diagram showing the assay plate according to one embodiment of the present invention;

FIGS. 3A to 3C are schematic diagrams showing the operation method of the assay plate according to one embodiment of the present invention;

FIGS. 4A to 4C are schematic diagrams showing the operation method of the assay plate according to one embodiment of the present invention;

FIG. 5 shows the movement situation of the droplets without shaking; and

FIG. 6 shows the movement situation of the droplets with shaking.

### DESCRIPTION OF THE PREFERRED EMBODIMENT

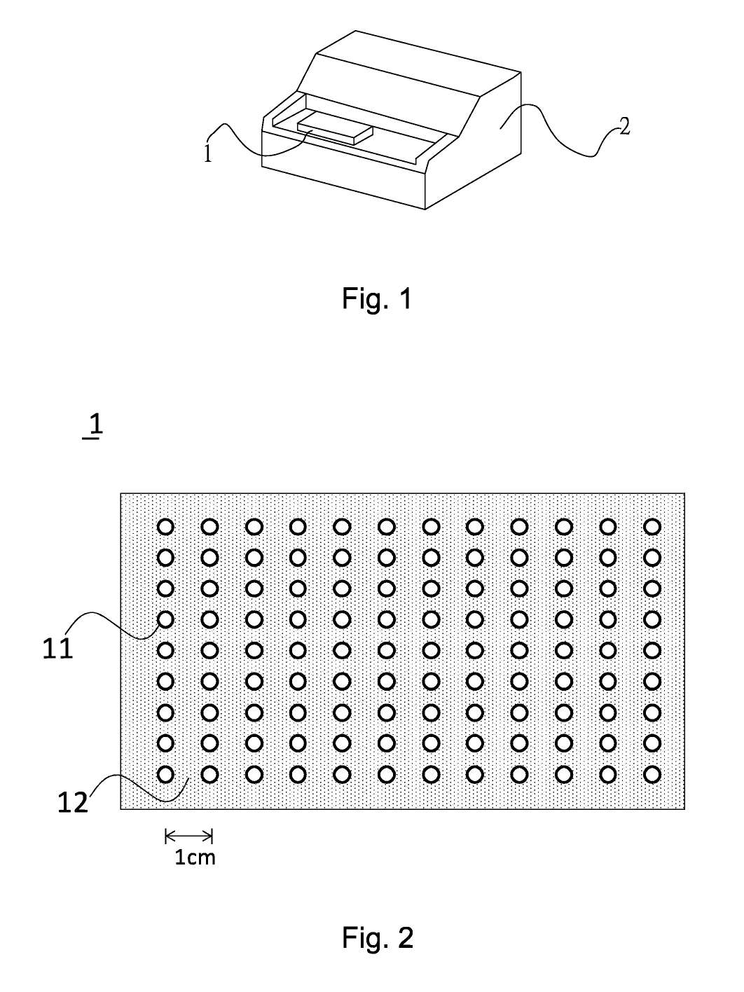

Please refer to FIG. 1 and FIG. 2, which are schematic diagrams showing the assay assembly and the assay plate according to one embodiment of the present invention. The assay assembly adopts the assay plate 1, which has at least one hydrophobic area 12 and a plurality of hydrophilic areas 11 defined by the hydrophobic area 12. The hydrophilic areas 11 are surrounded by the hydrophobic area 12, and each hydrophilic area 11 is independent respectively. The hydrophilic areas 11 are defined as a plurality of assay areas. By defining the hydrophilic areas 11 and the hydrophobic area 12, the assay plate 1 may have a result similar to the lotus effect to draw droplets around the assay areas into the assay area.

The size, amount and shape of the hydrophilic areas 11 in the assay plate 1 are not limited herein. As shown in FIG. 2, in one embodiment of the present invention, the assay plate 1 adopts the arrangement of a traditional 96-well plate, including the size and shape. The row spacing of the arrangement is about 1 cm. Besides, the assay plate 1 may also adopt the arrangement of 24-well plate or 384-well plate. The arrangement of the hydrophilic areas 11 is not supposed to be limited in the present invention and may be modified according to actual requirement.

Material of the hydrophilic areas 11 in the assay plate 1 is cellulose or synthetic polymer, and more specifically, is a porous and absorbent material. In one preferred embodiment, material of the hydrophilic areas 11 is filter paper or nitrocellulose membrane.

The chromatography filter paper adopted in the present invention is a semi-permeable test paper that is generally used for isolating the solid from the liquid or the air. Main material of the filter paper is plant fiber which generally gotten out from wood or cotton.

Wherein, one preferred embodiment is adopting the Whatman® cellulose chromatography filter papers, and its material is cotton fiber.

Furthermore, the absorption characteristics of the chromatography filter paper and the nitrocellulose membrane are different and may be accordingly adopted in the present invention. To specify, when observing the absorption characteristics, the nitrocellulose membrane tends to the surface adhesion which is generally used in transfer printing the biochemical material (such as protein), and the chromatography filter paper has a better water permeability and greater solute absorbability and is thus different from the nitrocellulose membrane.

Those skilled in the art may infer various methods to define the hydrophobic area 12 in the assay plate 1. For example, in one preferred embodiment of the present invention, the hydrophobic area 12 is manufactured by coating chemical material, such as wax printing.

In one embodiment, the chromatography filter paper may be patterned by wax printing, and then the patterned chromatography filter paper is heated on the baking tray (100° C., 10 mins) to obtain the chromatography filter paper plate in the present invention.

Those skilled in the art may also infer other manufacturing method to achieve the same purpose. In one embodiment, the SU-8 photoresist is coated and then irradiated by UV light to form the hydrophobic area 12, and therefore define the hydrophilic areas 11.

The assay area of the assay plate 1 may be transparent or opaque. When the assay area is transparent, the transparent assay method may be utilized to measure the transparence difference and obtain the reaction result.

When the assay area is opaque, the reflective assay method may be utilized to measure the reaction result.

The assay assembly of the present invention may be applied to measure the UV light, visible light, or fluorescent light, such as measuring, including but not being limited to, the biochemical reaction of ELISA.

Those skilled in the art may also infer other equipments or methods to aspirate droplets to the assay plate 1. The droplet aspirating equipment is instanced here, but not limited to, as Pipette, including 8-channel Pipette or other Multichannel Pipette. Besides, the robot for high speed screening may be also used to automatically aspirate and release droplets.

Please refer to FIG. 1 again, the assay plate 1 is coupled to a shaker 2 and the shaker 2 is configured for shaking the assay plate 1. Thereby droplets around the assay areas are further drawn to the assay areas by the shaking of the shaker 2, the pushing force of the hydrophobic area 12 and the pulling force of the assay areas.

The shaking direction of the shaker 2 may be designed according to actual requirements. For example, but not for limitation to, the direction may be vertical, horizontal, combined or random shaking. In one preferred embodiment, the shaking distance of the shaker 2 is shorter than half of the spacing of the assay areas in the assay plate 1. The repeated shaking may keep droplets effectively backing to the assay area. In one preferred embodiment, the shaker 2 may be the micro well shaker in the market.

In one preferred embodiment, the shaker 2 may be the micro well shaker in the market. Besides, the shaker 2 in the present invention may be the incubator or the reader as well.

Further, in one embodiment, the assay plate 1 is a single layer paper plate structure. The assay plate 1 may be equipped on a carrier (not shown), and be coupled to the shaker 2 through the carrier. In one preferred embodiment, the carrier is a 96-well plastic plate in the market.

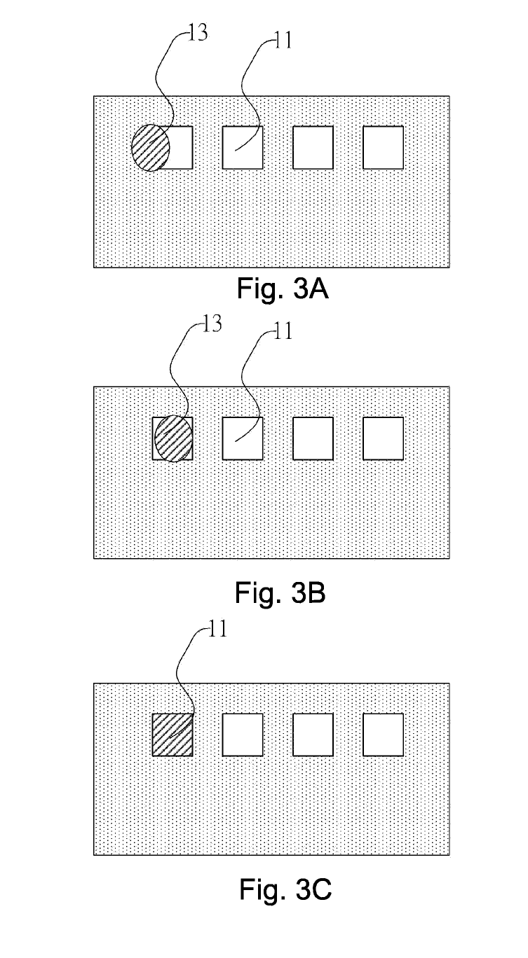

Please refer to FIGS. 3A to 3C for the operation method of the assay assembly of the present invention. The assay plate is fixed to the shaker, and then the Pipette aspirates the droplet 13 and release to the hydrophilic areas 11 of the assay plate. After a while, the droplets 13 around the hydrophilic areas 11 are drawn to the hydrophilic areas 11 by hydrophilic force of the hydrophilic areas 11.

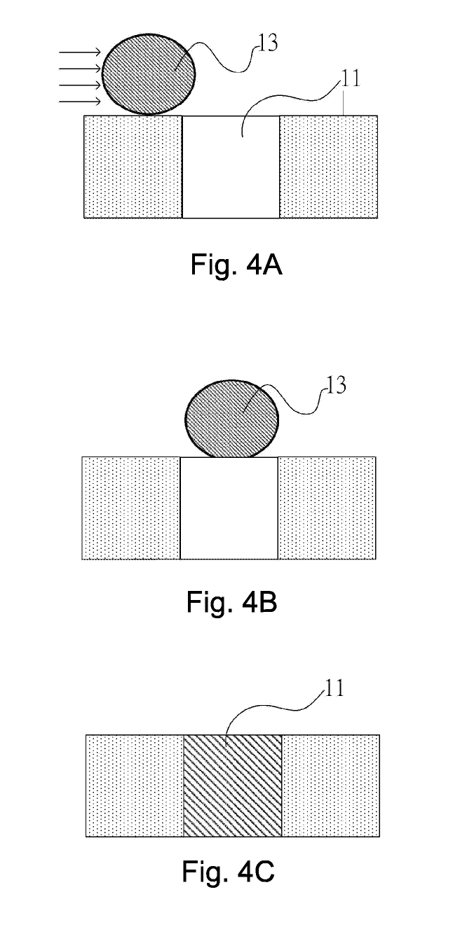

Please further refer to FIGS. 4A to 4C, with regard to the droplets not entering the assay area, the shaker 2 may continuously shake the assay plate 1 to make the droplets 13 around the hydrophilic areas 11 drawn to the hydrophilic areas 11 by hydrophilic force.

In order to make the objectives, technical solutions and advantages of the embodiments of the present invention clearer, the embodiments of the present invention are further described in detail below with reference to the embodiments and accompanying drawings. Here, the exemplary embodiments and the illustrations of the present invention are only intended to explain the present invention, rather than limit the present invention.

TABLE 1
The movement of droplets without shaking

| Group | (1) | (2) | (3) | (4) | (5) | (6) |
|---|---|---|---|---|---|---|
| Distance | 0 mm | −1 mm | 1 mm | 2 mm | 3 mm | 4 mm |
| between | | | | | | |
| the | | | | | | |
| assay | | | | | | |
| area | | | | | | |
| border | | | | | | |
| and the | | | | | | |
| center of | | | | | | |
| droplet | | | | | | |
| (mm) | | | | | | |
| Ratio of | 50% | 75% | 25% | 14% | 0% | 0% |
| the | | | | | | |
| droplet | | | | | | |
| area and | | | | | | |
| the | | | | | | |
| assay | | | | | | |
| area (%) | | | | | | |
| Droplet | 100% | 100% | 100% | 100% | 0% | 0% |
| move to | | | | | | |
| the | | | | | | |
| assay | | | | | | |
| area | | | | | | |
| (N = 12) | | | | | | |
| Residual | No | No | No | No | No | No |
| result | residual | residual | residual | residual | movement | movement |
| (N = 12) | | | | | of | of |
| | | | | | droplet | droplet |

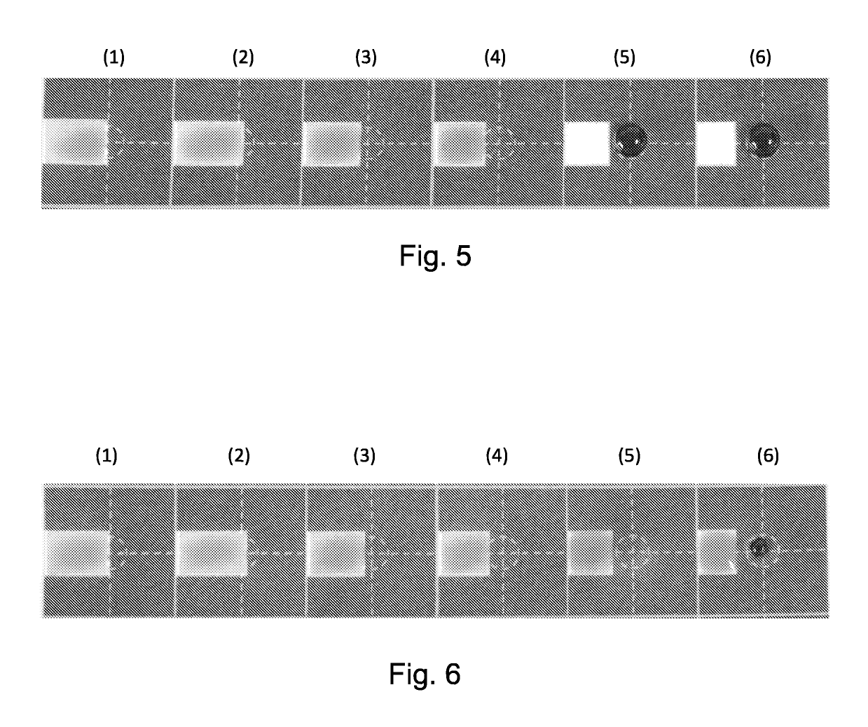

Please refer to Table 1 and FIG. 5 showing the movement situation of the droplets without shaking. For testing the error-tolerance rate of the water-soluble solvent on the paper imprint platform, the experiment of the distance between the droplet and the assay area is designed in the present invention. Without using the shaker, the distance is measured to determine how far the droplet may draw back to the assay area. The experiment condition is set to use 40 a red stain to fill the central circle area, and the diameter of the central circle area is 5 mm. When the distance between the assay area border and the center of droplet is 2 mm, the droplets may move to the assay area with the pushing force of the hydrophobic area and the pulling force of the assay areas, and no residual of the droplet. When the distance between the assay area border and the center of droplet is 3 mm, the droplets are no longer locating in the assay area, and no movement found.

TABLE 2
The movement of droplets with shaking

| Group | (1) | (2) | (3) | (4) | (5) | (6) |
|---|---|---|---|---|---|---|
| Distance | 0 mm | −1 mm | 1 mm | 2 mm | 3 mm | 4 mm |
| between | | | | | | |
| the assay | | | | | | |
| area | | | | | | |
| border | | | | | | |
| and the | | | | | | |
| center of | | | | | | |
| droplet | | | | | | |
| (mm) | | | | | | |
| Ratio of | 50% | 75% | 25% | 14% | 0% | 0% |
| the | | | | | | |
| droplet | | | | | | |
| area and | | | | | | |
| the assay | | | | | | |
| area (%) | | | | | | |
| Droplet | 100% | 100% | 100% | 100% | 100% | 100% |
| move to | | | | | | |
| the assay | | | | | | |
| area | | | | | | |
| (N = 12) | | | | | | |
| Residual | No | No | No | No | No | Droplet |
| result | residual | residual | residual | residual | residual | residual |
| (N = 12) | | | | | | |

Please refer to Table 2 and FIG. 6 that show the movement situation of the droplets with shaking (Using general Vortex-Genie 2 shaker of Scientific Industries, the shaking condition is rotating around circle, 600 RPM, 1 sec, 3 times). When the distance between the assay area border and the center of droplet is 3 mm, the droplets may move to the assay area with the effect of shaker, the pushing force of the hydrophobic area and the pulling force of the assay areas, and no residual of the droplet. When the distance between the assay area border and the center of droplet is 4 mm, the droplets may move to the assay area with the above-mention forces, but droplet residual is found. By comparing Table 1 and Table 2, it is understood that the shaking may effectively improve the movement of droplets and further improve the error-tolerance rate of the assay plate.

Conclusively speaking, the assay assembly of the present invention may improve the error-tolerance rate of the assay plate and be used for high-throughput screening through the effect of shaker, the pushing force of the hydrophobic area and the pulling force of the assay areas.

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. An assay assembly, comprising:
an assay plate provided with at least a hydrophobic area and a plurality of hydrophilic areas defined by the hydrophobic area, wherein the hydrophilic areas are defined as a plurality of assay areas; and
a shaker, wherein the assay plate is coupled to the shaker and the shaker is configured for shaking the assay plate, thereby droplets around the assay areas are further drawn to the assay areas by the shaking of the shaker, the pushing force of the hydrophobic area and the pulling force of the assay areas.

2. The assay assembly of claim 1, wherein the assay plate is a 96-well plate or a 384-well plate.

3. The assay assembly of claim 1, wherein the assay areas are transparent.

4. The assay assembly of claim 1, wherein the assay areas are opaque.

5. The assay assembly of claim 1, wherein material of the assay areas is cellulose or synthetic polymer.

6. The assay assembly of claim 1, wherein material of the assay areas is filter paper or nitrocellulose membrane.

7. The assay assembly of claim 1, wherein the hydrophobic area is prepared by wax printing.

8. The assay assembly of claim 1, wherein material of the hydrophobic area is SU-8 photoresist.

9. The assay assembly of claim 1, wherein the assay plate is a single layer paper plate structure.
